SEQUENCE LISTING (23 sequences)

SEQ ID NO: 1 - protein, 911 aa, Homo sapiens [sequence omitted]

SEQ ID NO: 2 - DNA, 2740 bp, Homo sapiens
  human anion exchange protein 1 (AE1), band 3 protein
cgccatggaggagctgcaggatgattatgaagacatgatggaggagaatctggagcaggaggaatatgaagacccagacatccccgagtcccagatggaggagccggcagctcacgacaccgaggcaacagccacagactaccacaccacatcacacccgggtacccacaaggtctatgtggagctgcaggagctggtgatggacgaaaagaaccaggagctgagatggatggaggcggcgcgctgggtgcaactggaggagaacctgggggagaatggggcctggggccgcccgcacctctctcacctcaccttctggagcctcctagagctgcgtagagtcttcaccaagggtactgttctcctagacctgcaagagacctccctggctggagtggccaaccaactgctagacaggtttatctttgaagaccagatccggcctcaggaccgagaggagctgctccgggccctgctgcttaaacacagccacgctggagagctggaggccctggggggtgtgaagcctgcagtcctgacacgctctggggatccttcacagcctctgctcccccaacactcctcactggagacacagctcttctgtgagcagggagatgggggcacagaagggcactcaccatctggaattctggaaaagattcccccggattcagaggccacgttggtgctagtgggccgcgccgacttcctggagcagccggtgctgggcttcgtgaggctgcaggaggcagcggagctggaggcggtggagctgccggtgcctatacgcttcctctttgtgttgctgggacctgaggccccccacatcgattacacccagcttggccgggctgctgccaccctcatgtcagagagggtgttccgcatagatgcctacatggctcagagccgaggggagctgctgcactccctagagggcttcctggactgcagcctagtgctgcctcccaccgatgccccctccgagcaggcactgctcagtctggtgcctgtgcagagggagctacttcgaaggcgctatcagtccagccctgccaagccagactccagcttctacaagggcctagacttaaatgggggcccagatgaccctctgcagcagacaggccagctcttcgggggcctggtgcgtgatatccggcgccgctacccctattacctgagtgacatcacagatgcattcagcccccaggtcctggctgccgtcatcttcatctactttgctgcactgtcacccgccatcaccttcggcggcctcctgggagaaaagacccggaaccagatgggagtgtcggagctgctgatctccactgcagtgcagggcattctcttcgccctgctgggggctcagcccctgcttgtggtcggcttctcaggacccctgctggtgtttgaggaagccttcttctcgttctgcgagaccaacggtctagagtacatcgtgggccgcgtgtggatcggcttctggctcatcctgctggtggtgttggtggtggccttcgagggtagcttcctggtccgcttcatctcccgctatacccaggagatcttctccttcctcatttccctcatcttcatctatgagactttctccaagctgatcaagatcttccaggaccacccactacagaagacttataactacaacgtgttgatggtgcccaaacctcagggccccctgcccaacacagccctcctctcccttgtgctcatggccggtaccttcttctttgccatgatgctgcgcaagttcaagaacagctcctatttccctggcaagctgcgtcgggtcatcggggacttcggggtccccatctccatcctgatcatggtcctggtggatttcttcattcaggatacctacacccagaaactctcggtgcctgatggcttcaaggtgtccaactcctcagcccggggctgggtcatccacccactgggcttgcgttccgagtttcccatctggatgatgtttgcctccgccctgcctgctctgctggtcttcatcctcatattcctggagtctcagatcaccacgctgattgtcagcaaacctgagcgcaagatggtcaagggctccggcttccacctggacctgctgctggtagtaggcatgggtggggtggccgccctctttgggatgccctggctcagtgccaccaccgtgcgttccgtcacccatgccaacgccctcactgtcatgggcaaagccagcaccccaggggctgcagcccagatccaggaggtcaaagagcagcggatcagtggactcctggtcgctgtgcttgtgggcctgtccatcctcatggagcccatcctgtcccgcatccccctggctgtactgtttggcatcttcctctacatgggggtcacgtcgctcagcggcatccagctctttgaccgcatcttgcttctgttcaagccacccaagtatcacccagatgtgccctacgtcaagcgggtgaagacctggcgcatgcacttattcacgggcatccagatcatctgcctggcagtgctgtgggtggtgaagtccacgccggcctccctggccctgcccttcgtcctcatcctcactgtgccgctgcggcgcgtcctgctgccgctcatcttcaggaacgtggagcttcagtgtctggatgctgatgatgccaaggcaacctttgatgaggaggaaggtcgggatgaatacgacgaagtggccatgcctgtgtga

SEQ ID NO: 3 - protein, 10 aa, Artificial Sequence
  Description of Artificial Sequenceresidues 536-545 of AE1 protein, predicted alpha-helix
TFSKLIKIFQ

SEQ ID NO: 4 - protein, 10 aa, Artificial Sequence
  Description of Artificial Sequencepeptide mimic when used as antigen raises antibodies which bind to and cause destruction of pathologically adherent erythrocytes
TFSXLIXIFQ

SEQ ID NO: 5 - protein, 14 aa, Artificial Sequence
  Description of Artificial Sequencepreferred native AE1 sequence, peptide including two residues on either side of predicted alpha-helix
YETFSKLIKIFQDH

SEQ ID NO: 6 - protein, 14 aa, Artificial Sequence
  Description of Artificial Sequencepeptide mimic when used as antigen raises antibodies which bind to and cause destruction of pathologically adherent erythrocytes
YXTFSXLIXIFQXX

SEQ ID NO: 7 - protein, 10 aa, Artificial Sequence
  Description of Artificial Sequenceexemplar peptide
TFSDLIEIFQ

SEQ ID NO: 8 - protein, 10 aa, Artificial Sequence
  Description of Artificial Sequenceexemplar peptide
TFSDLIDIFQ

SEQ ID NO: 9 - protein, 10 aa, Artificial Sequence
  Description of Artificial Sequenceexemplar peptide
TFSRLIHIFQ

SEQ ID NO: 10 - protein, 10 aa, Artificial Sequence
  Description of Artificial Sequenceexemplar peptide
TFSDLIKIFQ

SEQ ID NO: 11 - protein, 10 aa, Artificial Sequence
  Description of Artificial Sequenceexemplar peptide
TFSHLIEIFQ

SEQ ID NO: 12 - protein, 10 aa, Artificial Sequence
  Description of Artificial Sequenceexemplar peptide
TFSDLIEIFQ

SEQ ID NO: 13 - protein, 10 aa, Artificial Sequence
  Description of Artificial Sequenceexemplar peptide
TFSELIEIFQ

SEQ ID NO: 14 - protein, 10 aa, Artificial Sequence
  Description of Artificial Sequenceexemplar peptide
TFSRLIRIFQ

SEQ ID NO: 15 - protein, 10 aa, Artificial Sequence
  Description of Artificial Sequenceexemplar peptide
AFSKLIKIFQ

SEQ ID NO: 16 - protein, 10 aa, Artificial Sequence
  Description of Artificial Sequenceexemplar peptide
TYSDLIDIFQ

SEQ ID NO: 17 - protein, 10 aa, Artificial Sequence
  Description of Artificial Sequenceexemplar peptide
TFSKLIKLFQ

SEQ ID NO: 18 - protein, 10 aa, Artificial Sequence
  Description of Artificial Sequenceexemplar peptide
TFSDLIKIFN

SEQ ID NO: 19 - protein, 10 aa, Artificial Sequence
  Description of Artificial Sequenceexemplar peptide
TFSDLVDIFQ

SEQ ID NO: 20 - protein, 10 aa, Artificial Sequence
  Description of Artificial Sequenceexemplar peptide
TFARLIRLFQ

SEQ ID NO: 21 - protein, 10 aa, Artificial Sequence
  Description of Artificial Sequenceexemplar peptide
TFSELIKIWN

SEQ ID NO: 22 - protein, 9 aa, Artificial Sequence
  Description of Artificial Sequencepeptide fragment containing amino terminal portion of SEQ ID NO5 or 6
YETFSKLIK

SEQ ID NO: 23 - protein, 9 aa, Artificial Sequence
  Description of Artificial Sequencepeptide fragment containing amino terminal portion of SEQ ID NO5 or 6
YDTFSRLIR

(19) United States
(12) Patent Application Publication
(10) Pub. No.: US 2005/0059588 A1
(43) Pub. Date: Mar. 17, 2005

(54) Peptides which generate antibodies resulting in lysis of pathologically adherent erythrocytes

(75) Inventors: Irwin Sherman, Riverside, CA (US); Enrique Winograd, Claremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 10/663,215

(22) Filed: Sep. 15, 2003

(51) Int. Cl. A61K 38/17; A61K 38/10; A61K 38/08

(52) U.S. Cl. 514/12; 514/13; 514/14; 514/15

(57) ABSTRACT

The invention provides peptides and nucleic acids encoding them that can be used to raise antibodies that bind to and cause destruction of cells that have undergone conformational changes in human anion exchange protein 1 (AE1). The invention further provides methods for using the proteins and nucleic acids, uses for them, and compositions of the proteins or nucleic acids and a pharmaceutically acceptable carrier.

### STATEMENT AS TO RIGHTS TO INVENTIONS MADE UNDER FEDERALLY SPONSORED RESEARCH OR DEVELOPMENT

This invention was made with Government support under Grant No. AI 43608, awarded by the National Institute of Allergy and Infectious Diseases of the National Institutes of Health. The Government has certain rights in the invention.

### CROSS-REFERENCES TO RELATED APPLICATIONS

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention relates to compositions and methods for use in lysing pathologically adhesive red blood cells. In particular, the present invention is directed to compositions and methods for use in connection with various conditions characterized in part by red blood cells with pathologically increased adhesiveness, for example as a result of malaria, sickle cell disease, thalassemia or diabetes.

Sickle cell disease is a result of the presence of the altered gene product hemoglobin S. This disease is characterized by hemolytic anemia and complications resulting from episodic vaso-occlusive events, and despite the fact that more than 50 years have elapsed since the existence of a “vicious cycle” of sickling and erythrostasis was reported (Ham and Castle (1940) Trans. Assoc. Am. Physicians 55:127-132), there still remain significant gaps in understanding of the mechanisms whereby sickle hemoglobin leads to the various manifestations of this disorder. Although the tendency of hemoglobin S to polymerize with reduced oxygen tension is the fundamental abnormality in sickle cell disease, polymerization and sickling itself do not entirely explain the pathophysiology of this disorder. In particular, membrane alterations in the sickle red cell contribute to sickle cell disease. The well-recognized complications of this syndrome such as recurrent and episodic painful crises, ischemic damage to tissues and organs, increased infections, and stroke presumably result from local disturbances in blood flow. The debilitating episodes of sickle cell crisis have been difficult to manage other than with hydration and analgesia.

Investigations have centered on the interaction of the sickle red cell and the vascular endothelial cell, in an attempt to identify those factors that could provoke a delay in microvascular flow. It was found that sickle red cells had a higher degree of adhesiveness to cultured human vascular endothelial cells than normal cells, and that this required neither frank morphologic deformation of the cells nor deoxygenation (Hoover et al., (1979) Blood 54, 872-876; Hebbel, R. P. et al. (1980) J. Clin. Invest. 65, 154-160). This seminal observation with human umbilical vein endothelial cells was later confirmed both in static and flow systems using endothelial cells from a variety of tissues, and from mammalian sources other than humans. Significantly, among patients with sickle cell anemia, frequency of acute vaso-occlusive crises correlates with red blood cell adherence to endothelium. Accordingly, sickle cell adherence to endothelium was identified as the likely factor that initiates acute vaso-occlusion in sickle-cell disease, either by primarily occluding small vessels or by slowing microvascular blood flow so that secondary, reversible red blood cell sickling can occur.

Alterations in the surface of the sickle red cell involve changes in the distribution of surface charge, evidenced by the clustering of cationized ferritin on the surface of such cells; this was not found on cells containing normal hemoglobin (Hebbel & Eaton (1982) in Membranes and Genetic Disorders, A. R. Liss Inc., NY, pp. 311-349). Calcium loading of normal cells induced both endothelial adherence and surface clumping of cationized ferritin. The binding of sickle cells might thus involve a redistribution of proteins. The mechanisms that might underlie such redistribution, however, were not revealed.

In the normal red cell, the integral proteins glycophorin and human anion exchange protein 1 (“AE1,” sometimes also referred to as “band 3” protein) are randomly distributed in the membrane. Treatments of red cells to produce hemoglobin denaturation (hemichrome formation), ATP depletion, calcium loading, and oxidative cross-linking can all result in the formation of clusters of integral membrane proteins which may be visualized by freeze fracture electron microscopy. Clusters of intramembranous particles (composed principally of AE1 and glycophorin) are apparent at sites of brilliant cresyl blue induced hemichrome binding in α-thalassemic cells (Lessin, L. S. et al. (1972) Arch. Intern. Med. 129:306-319), in phenylhydrazine-treated cells (Low, P. S. (1989) in Hematology, Red Blood Cell Membranes: Structure, Function, Clinical Implications, Vol. 11, P. Agre & J. C. Parker, eds., Marcel Dekker, Inc., pp. 237-260), in erythrocytes that contain mature forms of P. falciparum (Allred, D. R. et al. (1986) J. Cell Science 81:1-16), and in irreversibly sickled cells (Lessin, L. S. et al. (1974) Proceedings of 1st Natl. Sympos. on Sickle Cell Disease, NIH, Bethesda, Md., pp. 213-214).

Sickle red cells generate excessive amounts of superoxide due to accelerated auto-oxidation of sickle heme (Hebbel et al. (1982) J. Clin. Inv. 70, 1253-1259). This oxidant damage affects cellular hydration, increases hemichrome levels, causes the formation of hemichrome-stabilized membrane protein aggregates within the cell, and enhances adhesiveness. These same phenomena can be simulated in normal red cells by calcium loading or by treatment with the oxidant phenazine methosulfate (Hebbel et al. (1989) Am. J. Physiol. 256:C579-C583). Further, free iron is non-randomly associated with the co-clusters of hemichrome and AE1 (Repka et al. (1993) Blood 82:3204-3210), and could provide additional oxidant stress, focus damage to the underlying membrane structure, and promote further local hemichrome formation.

Adhesiveness is also observed in malaria-infected red cells. The hallmark of P. falciparum infections is sequestration, that is, attachment of erythrocytes infected with the mature stages of the parasite to the endothelial cells lining the post-capillary venules. This occurs principally in the lung, kidney, liver, heart and brain (Aikawa, M. et al. (1990) Am. J. Trop. Med. Hygiene 43:30-37; Pongponratn, E. et al. (1991) Am. J. Trop. Med. Hygiene 44:168-175). Sequestration may totally occlude blood flow and result in tissue ischemia, coma, and death.

As with sickle and P. falciparum-infected erythrocytes the red blood cells from patients with diabetes have an abnormal adherence to the endothelium (Chappet, O. et al. (1994) Nouv. Rev. Fr. Hematol. 36: 281-288). The mortality and morbidity from diabetes are related to the vascular complications resulting from vasoocclusion as well as capillary damage, and more than 75% of diabetic patients die from vascular complications. One of the consequences of the high concentrations of glucose in the blood plasma is the non-enzymatic glycosylation (glycation) of a variety of proteins such as those of the red cell membrane as well as hemoglobin. The early glycation products undergo a slow series of chemical rearrangements to form irreversible advanced glycation end products (AGE) and these accumulate over the lifetime of the proteins, including those of the erythrocyte. The AGEs are potentially pathogenic, and bind to receptors on the endothelium. AE1 is easily accessible to glycation, and abnormal clustering of intramembranous particles has been shown for diabetic red cells (Rambini, R. et al. (1993) Membrane Biochemistry 10:71-80). Therefore, it is likely that alterations in the conformation of AE1, in concert with fibrinogen and to a lesser extent fibronectin, play a role in the enhanced adhesion of the red cell in diabetics.

The presence of sickle hemoglobin, hemoglobin S, is the underlying cause of sickle cell disease. The thalassemias are disorders of the red cell which involve a decreased synthesis of either of the protein chains of adult hemoglobin, hemoglobin A. This lack of coordination in synthesis leads to an accumulation of one chain relative to the other, and as a consequence the free chains aggregate and accumulate as insoluble inclusions at the inner face of the membrane, bound principally to AE1. Erythrocytes from patients with thalassemia bind to endothelial cells to a greater degree than do normal red cells, and such patients have greater risk of vascular occlusion. Addition of autologous platelet-rich plasma causes a further increase in the number of adherent thalassemic red cells (Butthep, P. et al. (1992) S. E. Asian J. Trop. Med. and Public Hlth. 23, suppl 2, 101-104). Thalassemic cells are enriched in calcium, the membranes of such cells when extracted with the non-ionic detergent Triton X-100 retain twice the amount of AE1 as does that of the normal red cell, and clusters of intramembranous particles (composed primarily of AE1 and glycophorin) are apparent at sites of brilliant cresyl blue induced hemichrome binding in thalassemic cells (Lessin, L. et al. (1972) Arch. Int. Med. 129:306-319). Despite the fact that thalassemia and sickle cell disease are due to different genes, in both syndromes the red cell is similarly altered.

Previous work in the laboratory of the present inventors resulted in the discovery that the otherwise unrelated conditions malaria, sickle cell disease, thalassemia, and diabetes resulted in modifications in AE1 changing the clustering conformation of the protein. As a consequence of this rearrangement, once cryptic adhesive sites become exposed. By this change in protein conformation and exposure, the normally non-adherent erythrocyte becomes a cell with enhanced endothelial adhesiveness. See, e.g., U.S. Pat. No. 6,124,262. These discoveries led to the discovery of peptides derived from cryptic portions of AE1 that could be used to reduce the adhesiveness of pathologically adherent erythrocytes. Id.

While the peptides disclosed in the '262 patent can be used to reduce adhesion of pathologically adhesive cells, such as red blood cells (“RBCs”) infected with P. falciparum, to vascular endothelial cells, use of the peptides to generate antibodies did not result in antibodies that caused lysis of the pathologically adhesive cells. It would be desirable to have peptides that could be used not only to reduce adhesion of pathologically adhesive RBCs to vascular endothelial cells, but which can also be used to raise antibodies that bind to and cause lysis of the RBC.

### BRIEF SUMMARY OF THE INVENTION

In one set of embodiments, the invention provides uses of an isolated peptide of 40 or fewer amino acids, comprising a sequence with at least 90% identity to a sequence TFSX₁LIX₂IFQ (SEQ ID NO:4), where X₁ and X₂ are independently selected from amino acids with a charge under physiological conditions, and wherein said peptide, when presented as an antigen, raises antibodies which bind to and cause destruction of pathologically adherent erythrocytes, for the manufacture of a medicament to cause destruction of erythrocytes that adhere to vascular endothelial cells due to a pathological condition. In some of these uses, X₁ and X₂ are both negatively charged and in some, X₁ and X₂ are both positively charged. In some embodiments, X₁ and X₂ are both lysine. One or more of the amino acids can be D-amino acids. In some uses, the peptide has the sequence TFSKLIKIFQ (SEQ ID NO:3). The pathological condition can be a member of the group consisting of diabetes, thalassemia, sickle cell anemia, and falciparum malaria. The medicament can comprise antibodies. The antibodies can be polyclonal or they can be monoclonal. If monoclonal, the antibodies can be humanized.

The invention further provides uses of nucleic acids encoding an isolated peptide of 40 or fewer amino acids, comprising a sequence at least 90% identical to a sequence TFSX₁LIX₂IFQ (SEQ ID NO:4), where X₁ and X₂ are independently selected from amino acids with a charge under physiological conditions, and wherein antibodies raised by said peptide bind to and cause destruction of pathologically adherent erythrocytes, for the manufacture of a medicament to cause destruction of erythrocytes that adhere to vascular endothelial cells due to a pathological condition. In some of these uses, X₁ and X₂ are both negatively charged and in some, X₁ and X₂ are both positively charged. In some embodiments, X₁ and X₂ are both lysine. The pathological condition can be selected from the group consisting of diabetes, thalassemia, sickle cell anemia, and malaria.

The invention further provides methods for destroying erythrocytes adherent due to a pathological condition, said method comprising administering to a patient with said adherent erythrocytes antibodies that specifically bind to a protein having an amino acid sequence YETFSKLIKIFQDH (SEQ ID NO:5) on said erythrocytes, wherein binding of said antibodies to said amino acid sequence results in destruction of the adherent erythrocytes. The pathological condition can be selected from the group consisting of diabetes, thalassemia, sickle cell anemia, and malaria.

The invention also provides methods for destroying erythrocytes adherent due to a pathological condition, said method comprising administering to a patient with said pathologically adherent erythrocytes an isolated peptide with at least 80% sequence identity to a sequence YX₁TFSX₂LIX₃IFQX₄X₅ (SEQ ID NO:6), or a fragment thereof, which peptide or fragment thereof, when presented as an antigen, raises antibodies which specifically bind to and cause lysis of said pathologically adherent erythrocytes, wherein X₁, X₂, X₃, X₄ and X₅ are independently selected from amino acids that bear a charge at physiological pH. In some of these methods, X₁ and X₄ bear the same charge and X₂ and X₃ bear the same charge, but the charge borne by X₁ and X₄ is not the same as the charge borne by X₂ and X₃. In some, the charge borne by X₂ and X₃ is positive. In some, X₂ and X₃ are lysine residues. The invention further provides methods wherein the peptide has 100% sequence identity to SEQ ID:6 and further wherein X₂ and X₃ are lysine residues, X₁ is a glutamic acid, X₄ is an aspartic acid and X₅ is a histidine (SEQ ID NO:5). One or more of said amino acids can be a D-amino acid.

In another set of embodiments, the invention provides methods for lysing erythrocytes adherent due to a pathological condition, said method comprising administering to a patient with said pathologically adherent erythrocytes a nucleic acid encoding a peptide with at least 80% sequence identity to the sequence YX₁TFSX₂LIX₃IFQX₄X₅ (SEQ ID NO:6), or fragment thereof which raises antibodies which specifically recognize said peptide, wherein X₁, X₂, X₃, X₄ and X₅ are independently selected from amino acids that bear a charge at physiological pH, wherein expression of said peptide raises antibodies which specifically bind to and cause lysis of said pathologically adherent erythrocytes. In some embodiments, X₁ and X₄ bear the same charge and X₂ and X₃ bear the same charge, but the charge borne by X₁ and X₄ is not the same as the charge borne by X₂ and X₃. In some of the methods, the charge borne by X₂ and X₃ is positive and in some, X₂ and X₃ are lysine residues. In some embodiments, the peptide has 100% sequence identity to SEQ ID:6 and further X₂ and X₃ are lysine residues, X₁ is a glutarnic acid, X₄ is an aspartic acid and X₅ is a histidine (SEQ ID NO:5).

The invention also provides compositions of an isolated peptide with at least 80% sequence identity to a sequence YX₁TFSX₂LIX₃IFQX₄X₅ (SEQ ID NO:6), wherein X₁, X₂, X₃, X₄ and X₅ are independently selected from amino acids that bear a charge at physiological pH, and wherein antibodies raised by said peptide bind to and cause destruction of pathologically adherent erythrocytes, and a pharmaceutically acceptable carrier. In some cases, X₁ and X₄ bear the same charge and X₂ and X₃ bear the same charge, but the charge borne by X₁ and X₄ is not the same as the charge borne by X₂ and X₃. In some instances, the charge borne by X₂ and X₃ is positive. In some, X₂ and X₃ are lysine residues. In some embodiments, the peptide has 100% sequence identity to SEQ ID NO:6 and X₂ and X₃ are lysine residues, X₁ is a glutamic acid, X₄ is an aspartic acid, and X₅ is a histidine (SEQ ID NO:5). One or more of the said amino acids can be a D-amino acid.

The invention further provides isolated peptides with at least 80% sequence identity to the sequence YX₁TFSX₂LIX₃IFQX₄X₅ (SEQ ID NO:6) or fragment thereof, which peptide or fragment, when presented as an antigen, raises antibodies that specifically bind to SEQ ID NO:5 and cause destruction of pathologically adherent erythrocytes and wherein X₁, X₂, X₃, X₄ and X₅ are independently selected from amino acids that bear a charge at physiological pH. In some embodiments, X₁ and X₄ can bear the same charge and X₂ and X₃ can bear the same charge, but the charge borne by X₁ and X₄ is not the same as the charge borne by X₂ and X₃. The charge borne by X₂ and X₃ can be positive; in some cases X₂ and X₃ are lysine residues. In some instances, the peptide has 100% sequence identity to SEQ ID NO:6 and X₂ and X₃ are lysine residues, X₁ is a glutamic acid, X₄ is an aspartic acid and X₅ is a histidine (SEQ ID NO:5). One or more of the amino acids can be a D-amino acid.

The invention fuirther provides isolated nucleic acids encoding a peptide with at least 80% sequence identity to YX₁TFSX₂LIX₃IFQX₄X₅ (SEQ ID NO:6) or a fragment thereof, which peptide or fragment, when presented as an antigen, raises antibodies that specifically bind to SEQ ID NO:5 and cause lysis of pathologically adherent erythrocytes and further wherein X₁, X₂, X₃, X₄ and X₅ are independently selected from amino acids that bear a charge at physiological pH. In some embodiments, X₁ and X₄ can bear the same charge and X₂ and X₃ can bear the same charge, but the charge borne by X₁ and X₄ is not the same as the charge borne by X₂ and X₃. The charge borne by X₂ and X₃ can be positive; in some cases X₂ and X₃ are lysine residues. In some instances, the encoded peptide has 100% sequence identity to SEQ ID NO:6 and X₂ and X₃ are lysine residues, X₁ is a glutamic acid, X₄ is an aspartic acid and X₅ is a histidine (SEQ ID NO:5). One or more of the amino acids can be a D-amino acid. Any of the isolated nucleic acids described above can be operably linked to a promoter.

The invention further provides compositions of an isolated nucleic acid encoding a peptide with at least 80% sequence identity to the sequence YX₁TFSX₂LIX₃IFQX₄X₅ (SEQ ID NO:6) or fragment thereof, which peptide or fragment, when presented as an antigen, raises antibodies that specifically bind to SEQ ID NO:5 and cause destruction of pathologically adherent erythrocytes, wherein XI, X₂, X₃, X₄ and X₅ are independently selected from amino acids that bear a charge at physiological pH, and a pharmaceutically acceptable carrier. In some embodiments, X₂ and X₃ are lysine residues, X₁ is a glutamic acid, X₄ is an aspartic acid, and X₅ is a histidine (SEQ ID NO:5).

### BRIEF DESCRIPTION OF THE DRAWINGS

FIG. 1. Detection of AE1 and PfEMP 1 on chymotrypsin treated IEs by Western blots. Lanes 1-3 monoclonal antibody IIE1 against AE1 (kindly provided by M. L. Jennings). Lanes 4-6 polyclonal antibody against the ATS (acidic terminal sequence) of PfEMP1 (kindly provided by M. Wahlgren). Control IEs (lanes 1, 4). Chymotrypsin digested IEs (lanes 2, 5). Digested uninfected erythrocytes (lanes 3, 6). CH65 is the chymotryptic cleavage product of band 3.

FIG. 2. Interaction of DBR peptide with CD36 transfected CHO cells. Cells were incubated for 1 hour at 37° C. with 40 μg/ml biotin labeled peptide, followed by 20 μg/ml Alexa-conjugated streptavidin.

FIG. 3. Concentration dependent inhibition of IE binding to CD36 by DBR peptide. Immobilized CD36 was incubated with the indicated concentrations of DBR peptide, and then a cytoadherence assay was conducted as shown in the legend of Table 1. Each value represents the mean ±SD of triplicate determinations.

FIG. 4. Recognition of the DBR peptide by immunoglobulins from individuals living in a malaria endemic region (“immunes”). Human sera from either control or “immunes” were added to human erythrocytes infected with P. falciparum (“IEs”) and after incubation, the surface bound immunoglobulins were eluted using isotonic glycine buffer pH 3.0. Biotin labeled peptides were immobilized on 96 well plates using streptavidin, and the specific binding of the surface eluted immunoglobulins to the peptides was determined using alkaline phosphatase conjugated anti-human IgG.

FIG. 5. Binding of anti-DBR antibodies to the surface of IEs by an indirect immunofluorescence assay. Live IEs were incubated with chicken polyclonal antibody against synthetic DBR peptide and after several washings with PBS, surface-bound immunoglobulins were detected using Alexa conjugated antibodies.

FIG. 6. Graph showing lysing of infected and uninfected erythrocytes by sera from rabbits immunized with DBR peptide, or from unimmunized rabbits. Rabbits (designated as “049” and “050”) were immunized with 200 μg of DBR peptide conjugated to KLH in complete Freund's adjuvant, followed by 5 booster injections of 100 μg conjugated peptide in incomplete Freund's adjuvant. Pre-immune (pre) or immune (immune) sera was diluted in PBS and incubated for 1 hr at 37° C. with either uninfected erythrocytes (nrbc) or with human erythrocytes infected with P. falciparum. Cells were centrifuged and the hemoglobin released into the supernatant was quantified by measuring the optical density at 405 nm.

FIG. 7. Predicted secondary structure of the DBR peptide. The central 10 amino acid core assumes a highly alpha-helical structure. The top line is a bar graph indicating the confidence of prediction. The next line down is a cartoon showing the position of the predicted helix. The third line down indicates with an “H” if the amino acid at the indicated position in the DBR peptide is predicted to be in an alpha-helix, or “C”, if the amino acid at the indicated position is predicted not to be in an alpha-helix. The following line sets out the sequence of the DBR peptide.

### DETAILED DESCRIPTION OF THE INVENTION

### Introduction

Surprisingly, peptides of human anion exchange protein 1 (“AE1,” SEQ ID NO:1) have been discovered that raise antibodies that cause lysis of cells with alterations in AE1 that result in pathologically adhesive erythrocytes. As set forth in the Background section, previous work from the laboratory of the present inventors has shown that certain pathological conditions cause alterations in the conformation of AE1. It was further shown that, in red blood cells, these alterations in conformation exposed normally cryptic portions of AE1, and that red blood cells exhibiting these cryptic portions of AE1 exhibited abnormally high adherence to CD36 molecules on the surface of vascular endothelial cells. See, U.S. Pat. No. 6,124,262 and Crandall et al., PNAS 90:4703-4707 (1993) (“Crandall 1993”). The previous work showed that peptides mimicking particular cryptic portions of AE1 could block binding of pathologically adherent erythrocytes to vascular endothelial cells. Id.

The peptides of the invention are a notable advance over previously disclosed peptides derived from AE1. Like the previously disclosed peptides, the peptides of the invention can be used to block binding of pathologically adherent erythrocytes to vascular endothelial cells, and therefore can be used to reduce the damage caused to tissue by vascular occlusion caused by such binding. Unlike the previously known peptides, the peptides of the invention also can be used to raise antibodies that bind to and cause lysis of pathologically adherent erythrocytes. The destruction of these abnormal erythrocytes can further reduce the occlusion of capillaries and other small blood vessels, and the consequent damage of that occlusion.

When antibodies were prepared in an animal model against an exemplar peptide of the invention, the antibodies lysed human red blood cells infected with P. falciparum (the parasite that causes falciparum malaria), but not uninfected red blood cells. The pre-immunization sera showed no reaction with either normal or infected red cells. The peptides of the invention can therefore be used as immunogens to create sera that react only with infected red cells, and which can destroy infected red cells. The immunized animals showed no anemia or other ill effects, indicating that the peptides could be used to protect against falciparum malaria or to enhance the immune response against it.

Some previous peptides derived from the AE1 protein encompass part of the carboxyl portion of the present peptides. For example, the peptides disclosed in the '262 patent and in Crandall 1993 encompass a portion of SEQ ID NO:5 Work with the previously known peptides, however, failed to result in antibodies that bind to and cause lysis of abnormally adherent erythrocytes. Similarly, a peptide that contains a portion of the carboxyl end of the present peptides were disclosed in U.S. Pat. No. 5,157,020. There is no indication in this patent that the advantages of including the residues from the amino terminus of the present peptides were recognized.

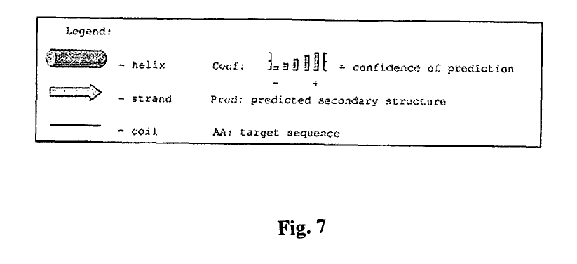

The present studies indicate that residues 536 to 545 of the AE1 protein (TFSKLIKIFQ, SEQ ID NO:5), form an alpha-helix, as shown in FIG. 7. Without wishing to be bound by theory, it appears that antibodies directed to the alpha helix formed by the amino acids of SEQ ID NO:5, and perhaps especially to epitopes that include the amino terminal end of SEQ ID NO:5, are particularly useful in marking for lysis cells with exposure of this normally cryptic portion of AE1.

A. Peptides and Nucleic Acids of the Invention

In one set of embodiments, the invention t provides isolated peptides comprising a sequence with at least 90% identity to the sequence TFSX₁LIX₂IFQ (SEQ ID NO:4) or fragment thereof, wherein the peptide, when used as an antigen, raises antibodies which specifically bind to and cause lysis of, pathologically adherent erythrocytes and where X₁ and X₂ are independently selected from amino acids that are charged under physiological conditions. These peptides mimic the sequence of a portion of the AE1 protein, residues 536 to 545 (TFSKLIKIFQ, SEQ ID NO:3), that is predicted to form an alpha-helix, as shown in FIG. 7.

As indicated in the sequence set forth above, the peptides of the invention encompass variants in which the “X” residues are any amino acids which bear a charge under physiological conditions. Thus, the peptides of the invention include such exemplar peptides as TFSDLIEIFQ (SEQ ID NO:7), TFSDLIDIFQ (SEQ ID NO:8), TFSRLIHIFQ (SEQ ID NO:9), TFSDLIKIFQ (SEQ ID NO: 10), TFSHLIEIFQ (SEQ ID NO: 11), TFSDLIEIFQ (SEQ ID NO:12), TFSELIEIFQ (SEQ ID NO:13) and TFSRLIRIFQ (SEQ ID NO:14). In general, peptides in which the two charged residues bear the same charge are preferred, with peptides in which the two charged residues are the same are more preferred. Peptides in which the charged residues are positively charged as particularly preferred. Any particular peptide can, of course, be tested to confirm whether it generates antibodies that bind to and cause lysis of cells with exposed normally cryptic portions of AE1 by assays known in the art, such as those taught in the Examples.

The peptides of the invention further include variants in which one non-charged residue of SEQ ID NO:3 is substituted with an amino acid that constitutes a conservative substitution. Conservative substitutions are considered in protein chemistry to be amino acids that have similar charge, steric and functional characteristics to another amino acid. A table of exemplar amino acids that are considered conservative substitutions of one another are set forth in Table B in the Definitions section below. Thus, for example, the peptides of the invention include such exemplar peptides as AFSKLIKIFQ (SEQ ID NO:15), TYSDLIDIFQ (SEQ ID NO:16), TFSKLIKLFQ (SEQ ID NO:17), TFSDLIKIFN (SEQ ID NO:18), TFSDLVDIFQ (SEQ ID NO:19), TFARLIRLFQ (SEQ ID NO:20), and TFSELIKIWN (SEQ ID NO:21). Any particular peptide can, of course, be tested to confirm whether it generates antibodies that bind to and cause lysis of cells with exposed normally cryptic portions of AE1 by assays known in the art, such as those taught in the Examples.

In a preferred form, the peptide comprises SEQ ID NO:4, and in a particularly preferred form, the peptide comprises the sequence of SEQ ID NO:3. The isolated peptides may be up to about 40 amino acids in length, with shorter peptides being preferred for ease of synthesis and administration. In general, peptides of 30 amino acids or fewer are more preferred, with about 20 amino acids being even more preferred. In a particularly preferred embodiment, the peptide is TFSKLIKIFQ, SEQ ID NO:3.

As noted, the peptides of the invention can be included in a longer isolated peptide totaling up to about 40 amino acids in length. Desirably, the peptides include the two residues present in AE1 on either side of the predicted alpha-helix of SEQ ID NO:3, as in the preferred native AE1 sequence YETFSKLIKIFQDH, SEQ ID NO:5. Of these four residues (two on either side of the predicted alpha-helix), three, the E, and D, and H, are charged. It is believed that one, two, or all three of these can be substituted with another charged residue without adversely affecting the ability of the peptide to generate antibodies which bind to and cause lysis of cells with an exposed SEQ ID NO:5 sequence. Thus, peptides of the sequence YX₁TFSX₂LIX₃IFQX₄X₅ (SEQ ID NO:6) can be used in the compositions and methods of the invention. Substitutions in which the substituted amino acid bears the same charge as the amino acid for which it is a substituent (as in substituting E for D or D for E) are preferred. Any particular peptide can, of course, be tested to confirm whether it generates antibodies that bind to and cause lysis of cells with exposed normally cryptic portions of AE1 by assays known in the art, such as those taught in the Examples.

Fragments of the peptides, particularly fragments containing the amino terminal portion of the peptide of SEQ ID NOS:5 or 6, can also be used. For example, YETFSKLIK (SEQ ID NO:21) or YDTFSRLIR (SEQ ID NO:22) may also be used. As noted, any particular peptide can be tested to confirm whether it generates antibodies that bind to and cause lysis of cells with exposed normally cryptic portions of AE1 by assays known in the art, such as those taught in the Examples.

One or more of the amino acids can be a D amino acid or can be modified to reduce proteolytic degradation of the peptide, increasing its life in the circulation.

The invention further provides nucleic acids encoding the peptides of the invention. Nucleic acids encoding the above described peptides, for example, can be readily determined by practitioners using standard genetic code tables. Due to the degeneracy of the genetic code, practitioners will appreciate that many nucleic acid molecules could be constructed to encode any given peptide of the invention. Preferably, the nucleic acids encoding a peptide of the invention are operably linked to a promoter to facilitate expression of the encoded peptide.

B. Uses of the Peptides and Nucleic Acids of the Invention

Red blood cells (“RBCs”) are not normally “sticky.” As noted in the Background section, RBCs become sticky in four otherwise unrelated pathological conditions, malaria, diabetes, thalassemia, and sickle cell anemia. It is believed that the change in stickiness, or adhesiveness, of the RBCs is due to changes in the conformation of AE1 which result in the exposure of otherwise cryptic portions of the AE1 protein. Whether or not an erythrocyte is pathologically adherent can be determined by standard assays, such as those taught in the Examples, below.

The peptides of the invention have multiple uses. In one set of embodiments, they can be used as immunogens to raise antibodies in animals. The antibodies can then be used in vitro to detect the presence of cells with distortions in the conformation of AE1 that expose the normally cryptic sequence of SEQ ID NO:3 This can be used, for example, to detect the presence of erythrocytes infected with P. falciparum, or of RBCs damaged by diabetes, by thalassemia, or by sickle cell anemia. The antibodies can further be used for passive immunization of a subject with or at risk of developing cells with changes in the conformation of AE1 which result in the exposure of otherwise cryptic portions of the AE1 protein, such as erythrocytes that are pathologically adherent. By binding and causing the lysis of such cells, tne antibodies can reduce the damage that would otherwise result from vascular occlusion caused by these cells.

Further, the peptides can be administered to subjects with, or at risk of developing, cells with changes in the conformation of AE1 which result in the exposure of otherwise cryptic portions of the AE1 protein. For example, the peptides can be administered to persons with, or at risk of developing, P. falciparum infection, diabetes, thalassemia, or sickle cell anemia. The peptides have two beneficial effects. First, as with the peptides disclosed in Crandall 1993, the peptides of the invention can bind to CD36 on the vascular endothelial cells, blocking binding of pathologically adherent RBCs to those cells. In studies in RBCs infected with P. falciparum, Crandall 1993, the investigators found that the peptides did not result in release of RBCs already bound to vascular endothelial cells, but prevented or reduced further cells from binding after administration of the peptides. Moreover, following administration of the peptides, parasitized cells appeared in the peripheral circulation, whereas in animals in which the peptides were not administered, such cells remained sequestered in the brain and other less accessible body compartments. This rendered the parasitized cells more accessible to anti-malarial chemotherapeutic agents.

Secondly, the peptides of the present invention are expected to raise antibodies that cause lysis of cells with changes in the conformation of AE1 which result in the exposure of otherwise cryptic portions of the AE1 protein, such as erythrocytes that are pathologically adherent. Without wishing to be bound by theory, it is believed that the peptides result in antibodies that, upon binding, either mark the cells for complement-mediated destruction or mark the cells for antibody-dependent cell-mediated cytotoxicity by neutrophils, cytotoxic T cells, or other cells of the immune system. Thus, the peptides of the invention can be used to enhance an immune response to conditions that result in such cells, such as P. falciparum infection, diabetes, thalassemia, and sickle cell anemia. The antibodies are expected to reduce the damage done to tissues due to vascular occlusion caused by abnormally adherent erythrocytes. The peptides can further be administered to enhance an immune response to any condition in which changes in the conformation of AE1 result in the exposure of otherwise cryptic portions of the AE1 protein.

If desired, nucleic acids encoding peptides of the invention can be administered in place of the peptides. Administration of nucleic acids to result in the expression of an encoded peptide is known in the art.

### Definitions

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

Human anion exchange protein 1 (“AE1,” also known in the art as the “band 3” protein) is present in a million copies per red cell in the form of monomers, dimers, or tetramers. Its molecular weight is approximately 95,000 kDa. There are two distinct domains: a 43 kDa water-soluble cytoplasmic domain, and a 55 kDa membrane-spanning domain (Low, P. S. (1986) Biochimica et Biophysica Acta 864, 145-167). The gene encoding AE1 was cloned and sequenced in the 1980's (Tanner, M. J. et al (1988) Biochem. J. 256, 703-12; Lux, S. E. et al. (1989) Proc. Nat'l Acad. Sci. USA 86, 9089-93). The deduced amino acid sequence (SEQ ID NO:1) and the nucleotide sequence (SEQ ID NO:2) encoding the amino acid sequence can be found in the Entrez database maintained by the National Center for Biotechnology Information at either of two accession numbers: X12609 and M27819.

“Pathologically adherent” cells refers to cells that have undergone changes in the conformation of AE1 which result in the exposure of otherwise cryptic portions of the AE1 protein. As used herein, the term usually refers to erythrocytes that are pathologically adherent due to P. falciparum infection, diabetes, thalassemia, or and sickle cell anemia. Whether or not a particular cell is pathologically adherent can be determined by standard assays, including the assays set forth in the Examples below.

“CD36” is a leukocyte differentiation antigen whose gene is found at 7q11.2. It is an 88 kD adhesion molecule that plays a major role as a scavenging receptor implicated in cellular lipid metabolism. CD36 is also called GP IIIb and functions as a thrombospondin receptor on platelets and monocytes and as receptor for Plasmodium falciparum infected erythrocytes. It is expressed in secretory mammary epithelium, in microvasculature endothelium, in adipocytes, in smooth muscle cells, and in platelets. Normal RBCs do not bind to CD36.

“DBR” refers to “DIDS binding region.” DIDS (4,4′-diisothiocyanatostilbene-2,2′-disulfonic acid) is a bifunctional inhibitor of anion exchange in erythrocytes and reacts covalently with Lys-539 in AE1. As used herein, DBR refers to peptides of SEQ ID NOS:3 or 5.

By “cell membrane proximal” or “cell surface proximal” is meant next to or nearer the cell membrane.

By “cell membrane distal” or “cell surface distal” is meant farther away from the cell membrane.

By “extracellular” is meant the region extending outward from the lipid bilayer encompassing a cell.

The term “epitope” refers to the portion of an antigen that is recognized and bound by an antibody.

By “specifically bind” is meant that no more than 15% of a ligand which specifically binds to a target molecule is bound to a particular non-target molecule. More preferably, no more than 10% is bound to the non-target molecule, even more preferably less than 5%, and most preferably less than 1%.

By “physiological conditions” is meant an extracellular milieu having conditions (e.g., temperature, pH, and osmolarity) which allows for the sustenance or growth of a cell of interest.

Cells are generally understood in the art to be bounded by a plasma membrane (herein referred to as the “cell membrane”) comprising a lipid bilayer, in which proteins such as the AE1 protein are situated. See, generally, Alberts et al., Molecular Biology of the Cell, Garland Publishing, Inc., New York (3rd Ed., 1994), Chapter 10. The cell membrane may be considered to have a surface facing on the cytosol, or the interior of the cell, and a surface facing to the exterior of the cell, or the extracellular space. Transmembrane proteins such as AE1 are amphipathic, that is, they have regions that are hydrophobic and regions that are hydrophilic. Regions that pass through the membrane are hydrophobic and interact with the hydrophobic tails of the lipid molecules comprising the bilayer. Regions that are hydrophilic are exposed to water on either the cytosolic or the extracellular side of the membrane. Id. The transmembrane domain of transmembrane proteins are either in an alpha helix or multiple beta strands. Lodish et al., Molecular Cell Biology, W. E. Freeman and Co., New York (4th Ed., 2000), at chapter 3.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies), heteroconjugate antibodies (e.g., bispecific antibodies) and recombinant single chain Fv fragments (scFv), and disulfide stabilized (dsFv) Fv fragments. The term “antibody” also includes antigen binding forms or fragments of antibodies which retain antigen binding capability (e.g., Fab′, F(ab′)2, Fab, Fv and rIgG). See also, Pierce Catalog and Handbook, 1994-1995 (Pierce Chemical Co., Rockford, Ill.); Kuby, J., Immunology, 3rd Ed., W. H. Freeman & Co., New York (1997).

An antibody immunologically reactive with a particular antigen can be generated by recombinant methods such as selection of libraries of recombinant antibodies in phage or similar vectors, see, e.g., Huse, et al., Science 246:1275-1281 (1989); Ward, et al., Nature 341:544-546 (1989); and Vaughan, et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined. See, Kabat, E., et al., SEQUENCES OF PROTEINS OF IMMUNOLOGICAL INTEREST, U.S. Government Printing Office, NIH Publication No. 91-3242 (1991). The Kabat and Wu database is the most widely used system in the art for numbering amino acid residues of antibodies and is now too large to be conveniently printed. It is now maintained online and can be found by entering “http://” followed by “immuno.bme.nwu.edu/”. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” or a “VH” refer to the variable region of an immunoglobulin heavy chain, including an Fv, scFv, dsFv or Fab. References to “VL” or a “VL” refer to the variable region of an immunoglobulin light chain, including of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

The term “contacting” includes reference to placement in direct physical association.

An “expression plasmid” comprises a nucleotide sequence encoding a molecule or interest, which is operably linked to a promoter.

As used herein, “polypeptide”, “peptide” and “protein” are used interchangeably and include reference to a polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

The term “residue” or “amino acid residue” or “amino acid” includes reference to an amino acid that is incorporated into a protein, polypeptide, or peptide (collectively “peptide”). The amino acid can be a naturally occurring amino acid and, unless otherwise limited, can encompass known analogs of natural amino acids that can function in a similar manner as naturally occurring amino acids.

The amino acids and analogs referred to herein are described by shorthand designations as follows in Table A:

TABLE A
Amino Acid Nomenclature

| Name | 3-letter | 1-letter |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Homoserine | Hse | — |
| Isoleucine | Ile | I |
| Leucine | Leu | L |
| Lysine | Lys | K |
| Methionine | Met | M |
| Methionine sulfoxide | Met (O) | — |
| Methionine | Met (S—Me) | — |
| methylsulfonium | | |
| Norleucine | Nle | — |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution”, when describing a protein refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups in Table B each contain amino acids that are conservative substitutions for one another:

TABLE B

1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

See also, Creighton, PROTEINS, W.H. Freeman and Company, New York (1984).

The terms “substantially similar” in the context of a peptide indicates that a peptide comprises a sequence with at least 90%, preferably at least 95% sequence identity to the reference sequence over a comparison window of 10-20 amino acids. Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The phrase “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond. In the context of this invention, the cysteines which form the disulfide bond are within the framework regions of the single chain antibody and serve to stabilize the conformation of the antibody.

The terms “conjugating,” “joining,” “bonding” or “linking” refer to making two polypeptides into one contiguous polypeptide molecule. In the context of the present invention, the terms include reference to joining an antibody moiety to an effector molecule (EM). The linkage can be either by chemical or recombinant means. Chemical means refers to a reaction between the antibody moiety and the effector molecule such that there is a covalent bond formed between the two molecules to form one molecule.

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, underexpressed or not expressed at all.

As used herein, “nucleic acid” or “nucleic acid sequence” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof as well as conservative variants, i.e., nucleic acids present in wobble positions of codons and variants that, when translated into a protein, result in a conservative substitution of an amino acid.

As used herein, “encoding” with respect to a specified nucleic acid, includes reference to nucleic acids which comprise the information for translation into the specified protein. The information is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Proc. Nat'l Acad. Sci. USA 82:2306-2309 (1985), or the ciliate Macronucleus, may be used when the nucleic acid is expressed in using the translational machinery of these organisms.

The phrase “fusing in frame” refers to joining two or more nucleic acid sequences which encode polypeptides so that the joined nucleic acid sequence translates into a single chain protein which comprises the original polypeptide chains.

As used herein, “expressed” includes reference to translation of a nucleic acid into a protein. Proteins may be expressed and remain intracellular, become a component of the cell surface membrane or be secreted into the extracellular matrix or medium.

By “host cell” is meant a cell which can support the replication or expression of the expression vector. Host cells may be prokaryotic cells such as E. coli, or eukaryotic cells such as yeast, insect, amphibian, or mammalian cells.

The phrase “phage display library” refers to a population of bacteriophage, each of which contains a foreign cDNA recombinantly fused in frame to a surface protein. The phage display the foreign protein encoded by the cDNA on its surface. After replication in a bacterial host, typically E. coli, the phage which contain the foreign cDNA of interest are selected by the expression of the foreign protein on the phage surface.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, more preferably 65%, even more preferably 70%, still more preferably 75%, even more preferably 80%, and most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (and can be found online by entering “http://www.”, followed by “ncbi.nlm.nih.gov/”). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The term “in vivo” includes reference to inside the body of the organism from which the cell was obtained. “Ex vivo” and “in vitro” means outside the body of the organism from which the cell was obtained.

As used herein, “mammalian cells” includes reference to cells derived from mammals including humans, rats, mice, guinea pigs, chimpanzees, or macaques. The cells may be cultured in vivo or in vitro.

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See Harlow & Lane, supra, for a description of immunoassay formats and conditions. Preferably, the immunologically reactive conditions employed in the methods of the present invention are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

### Generating Antibodies to Peptides of the Invention

Antibodies, including polyclonal, monoclonal, or recombinant single chain Fv antibodies, can be generated or constructed for use in the present invention. Methods of producing polyclonal and monoclonal antibodies are known to those of skill in the art. See, e.g., Coligan (1991) Current Protocols in Immunology Wiley/Greene, N.Y.; and Harlow and Lane (1989) Antibodies: A Laboratory Manual Cold Spring Harbor Press, N.Y.; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497; Huse et al. (1989) Science 246: 1275-1281; Ward, et al. (1989) Nature 341: 544-546. Birch and Lennox, Monoclonal Antibodies: Principles and Applications, Wiley-Liss, N.Y., N.Y. (1995).

Other suitable techniques for antibody or peptide ligand preparation include selection of libraries of recombinant antibodies/peptides in phage or similar vectors. High affinity antibodies and peptides to the stalk can be rapidly isolated by using phage display methods to express recombinant single chain Fv (scFv) fragments or peptide ligands on the phage surface. Briefly, genes encoding the surface protein of a phage are altered so as to allow the insertion of an antibody or peptide gene which is expressed as a fusion protein on the surface of the phage that carries the gene. The phage expressing the desired antibody or peptide ligand can be selectively enriched and isolated by virtue of its affinity/avidity for the stalk. The DNA encoding the ligand is packaged in the same phage and which allows the gene encoding the ligand to be isolated. A variety of such methods are amply discussed in the literature and well known to the skilled artisan. See, e.g., Winter et al., Annu. Rev. Immunol. 12:433-455 (1994); Marks et al., J. Mol Biol. 222:581-597 (1991); Vaughan et al., Nature Biotechnology 14:309-314 (1996), U.S. Pat. Nos. 4,642,334; 4,816,397; 4,816,567; 4,704,692; WO 86/01533; WO 88/09344; WO 89/00999; WO 90/02809; WO 90/04036; EP 0 324 162; EP 0 239 400.

In chemical peptide synthesis, a procedure termed “Divide, Couple and Recombine” (DCR) has been used to produce combinatorial peptide libraries. See, Furka et al, Int. J. Pept. Protein Res. 37:487-493 (1991) and Houghten et al., Nature 354:84-86 (1991). As an alternative to DCR, peptide mixtures have also been made by direct coupling of monomer mixtures. See, Rutter et al., U.S. Pat. No. 5,010,175. The use of such methods to produce mixtures of other linear polymers, such as “peptoids”, has been suggested. See, Simon, et al, Proc. Natl. Acad. Sci. USA 89:9367-9371 (1992). In oligonucleotide synthesis, “degenerate” or “wobble” mixtures of oligonucleotide products can be made by, for example, delivery of equimolar mixtures of monomers to an oligonucleotide polymer at specific steps during synthesis. See, Atkinson and Smith, in “Oligonucleotide Synthesis. A Practical Approach”, 1984, IRL Press, Oxford, edited by M. Gait, pp 35-81. These methods of synthesizing peptides or oligonucleotides provide large numbers of compounds for testing which, if active, can be readily identified.

Preferably, antibodies are generated or designed to minimize immunogenicity in the host as, for example, by maximizing the number of autologous (self) sequences present in the ligand. Accordingly, antibodies having non-xenogenic variable regions are preferred. Particularly preferred are the use of antibodies in which xenogenic portions are excluded, or are essentially limited to the complementarity determining regions, as in humanized antibodies, in which the Fc portion of, for example, a murine antibody is replaced with a human sequence. Techniques for humanizing antibodies are well known and the subject of multiple patents.

The antibodies can be detected and/or quantified using any of a number of well recognized immunological binding assays (see, e.g., U.S. Pat. Nos. 4,366,241; 4,376,110; 4,517,288; and 4,837,168). For a review of the general immunoassays, see also METHODS IN CELL BIOLOGY, VOL. 37, Asai, ed. Academic Press, Inc. New York (1993); BASIC AND CLINICAL IMMUNOLOGY 7TH EDITION, Stites & Terr, eds. (1991). Immunological binding assays (or immunoassays) typically utilize a ligand (e.g., a peptide of SEQ ID NO:3 or 5) to specifically bind to and often immobilize an antibody.

Binding affinity of antibodies to a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (KD=1/K, where K is the affinity constant) of the antibody is <1 μM, preferably <100 nM, and most preferably <0.1 nM. Antibody molecules will typically have a KD in the lower ranges. KD=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

Immunoassays also often utilize a labeling agent to specifically bind to and label the binding complex formed by the ligand and the antibody. The labeling agent may itself be one of the moieties comprising the antibody/analyte complex, i.e., an anti-SEQ ID NO:5 antibody). Alternatively, the labeling agent may be a third moiety, such as another antibody, that specifically binds to the antibody/SEQ ID NO:5 complex.

In one aspect, a competitive assay is contemplated wherein the labeling agent is a second antibody, such as an anti-SEQ ID NO:5 antibody, bearing a label. The two antibodies then compete for binding to the immobilized ligand, such as SEQ ID NO:5 peptide. Alternatively, in a non-competitive format, the first antibody lacks a label, but a second antibody specific to antibodies of the species from which the first antibody is derived, e.g., mouse, and which binds the first antibody, is labeled.

Other proteins capable of specifically binding immunoglobulin constant regions, such as Protein A or Protein G may also be used as the label agent. These proteins are normal constituents of the cell walls of streptococcal bacteria. They exhibit a strong non-immunogenic reactivity with immunoglobulin constant regions from a variety of species (see, generally Kronval, et al., J. Immunol. 111:1401-1406 (1973); and Akerstrom, et al., J. Immunol. 135:2589-2542 (1985)).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antibody, volume of solution, concentrations, and the like. Usually, the assays will be carried out at ambient temperature, although they can be conducted over a range of temperatures, such as 10° C. to 40° C. 0

While the details of the immunoassays of the present invention may vary with the particular format employed, the method of detecting antibodies of interest in a sample containing the antibodies generally comprises the steps of contacting the sample with an antibody which specifically reacts, under immunologically reactive conditions, to the ligand/antibody complex.

### Production of Nucleic Acids and Expression of Encoded Peptides of the Invention

A. Recombinant Methods

Nucleic acid sequences encoding peptides of the present invention can be prepared by any suitable method including, for example, cloning of appropriate sequences or by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Lett. 22:1859-1862 (1981); the solid phase phosphoramidite triester method described by Beaucage & Caruthers, Tetra. Letts. 22(20):1859-1862 (1981), e.g., using an automated synthesizer as described in, for example, Needham-VanDevanter, et al. Nucl. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In a preferred embodiment, the nucleic acid sequences of this invention are prepared by cloning techniques. Examples of appropriate cloning and sequencing techniques, and instructions sufficient to direct persons of skill through many cloning exercises are found in Sambrook, et al., supra, Berger and Kimmel (eds.), supra, and Ausubel, supra. Product information from manufacturers of biological reagents and experimental equipment also provide useful information. Such manufacturers include the SIGMA chemical company (Saint Louis, Mo.), R&D systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Nucleic acids encoding peptides of the invention can be modified to form variants of the peptides. Modification by site-directed mutagenesis is well known in the art. Nucleic acids encoding the peptides can be amplified by in vitro methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In a preferred embodiment, peptides of the invention are prepared by inserting cDNA which encodes the peptide into a vector. The insertion is made so that the peptide is read in frame, that is in one continuous polypeptide.

Once nucleic acids encoding a peptide of the invention are isolated and cloned, one may express the desired protein in a recombinantly engineered cell such as bacteria, plant, yeast, insect or mammalian cells. It is expected that those of skill in the art are knowledgeable in the numerous expression systems available for expression of proteins including E. Coli, other bacterial hosts, yeast, and various higher eukaryotic cells such as the COS, CHO, HeLa and myeloma cell lines.

One of skill would recognize that modifications can be made to a nucleic acid encoding a polypeptide of the present invention without diminishing its biological activity. Some modifications may be made to facilitate the cloning, expression, or incorporation of the targeting molecule into a fusion protein. Such modifications are well known to those of skill in the art and include, for example, termination codons, a methionine added at the amino terminus to provide an initiation site, and additional amino acids placed on either terminus to create conveniently located restriction sites.

In addition to recombinant methods, peptides of the present invention can also be constructed in whole or in part using standard peptide synthesis. Solid phase synthesis of the polypeptides of the present invention of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, THE PEPTIDES: ANALYSIS, SYNTHESIS, BIOLOGY. VOL. 2: SPECIAL METHODS IN PEPTIDE SYNTHESIS, PART A. pp. 3-284; Merrifield, et al. J. Am. Chem. Soc. 85:2149-2156 (1963), and Stewart, et al., SOLID PHASE PEPTIDE SYNTHESIS, 2ND ED., Pierce Chem. Co., Rockford, Ill. (1984). Proteins of greater length may be synthesized by condensation of the amino and carboxyl termini of shorter fragments. Methods of forming peptide bonds by activation of a carboxyl terminal end (e.g., by the use of the coupling reagent N,N′-dicycylohexylcarbodiimide) are known to those of skill.

B. Purification

Once expressed, peptides of the present invention can be purified according to standard procedures of the art, including ammonium sulfate precipitation, affinity columns, column chromatography, and the like (see, generally, R. Scopes, PROTEIN PURIFICATION, Springer-Verlag, N.Y. (1982)). Substantially pure compositions of at least about 90 to 95% homogeneity are preferred, and 98 to 99% or more homogeneity are most preferred for pharmaceutical uses. Once purified, partially or to homogeneity as desired, if to be used therapeutically, the polypeptides should be substantially free of endotoxin.

Antibodies to the peptides can also be expressed and purified by known techniques. Methods for expression of antibodies and/or refolding to an appropriate active form, including single chain antibodies, from bacteria such as E. coli have been described and are well-known. See, e.g., Buchner, et al., Anal. Biochem. 205:263-270 (1992); Pluckthun, Biotechnology 9:545 (1991); Huse, et al., Science 246:1275 (1989) and Ward, et al., Nature 341:544 (1989).

### Compositions of Peptides and Nucleic Acids of the Invention and Administration Thereof

In another aspect, this invention provides compositions that comprise a composition of this invention in a pharmaceutically acceptable carrier.

In one group of embodiments, the composition comprises a peptide of the invention in an amount effective to elicit a humoral immune response in a subject. Such compositions are useful in stimulating or enhancing an immune response to pathological conditions characterized by adherent erythorcytes. These compositions are preferably administered intradermally, subcutaneously, or intramuscularly.

The compositions for administration will commonly comprise a solution of the peptide dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of peptide in these formulations can vary widely, and will be selected primarily based on fluid volumes, viscosities, body weight and the like in accordance with the particular mode of administration selected.

The compositions of the invention are administered to a patient in an amount sufficient to elicit an effective immune response, preferably a humoral response, to adherent erythrocytes. An amount adequate to accomplish this is defined as “therapeutically effective dose.”

Amounts effective for this use will depend on, e.g., the peptide and/or protein composition, the manner of administration, the stage and severity of the pathological condition resulting in the adherent erythrocytes, the weight and general state of health of the patient, and the judgment of the prescribing physician, but generally range for the initial immunization (for therapeutic or prophylactic administration) from about 0.001 to about 200 mg/kg, more preferably about 0.01 to about 100 mg/kg, most preferably about 0.1 to 50 mg/kg peptide. The initial immunization may be followed by boosting dosages of from about 0.001 to about 100 mg/kg, more preferably about 0.01 to about 50 mg/kg peptide pursuant to a boosting regimen over weeks to months, depending upon the patient's response and condition determined by measuring levels of specific antibodies that react with peptides of the invention in the patient's blood.

Actual methods for preparing administrable compositions will be known or apparent to those skilled in the art and are described in more detail in such publications as REMINGTON'S Pharmaceutical SCIENCE, 19TH ED., Mack Publishing Company, Easton, Pa. (1995).

In another embodiment, the composition comprises isolated nucleic acid molecules comprising a nucleotide sequence encoding a peptide of the invention, which nucleic acid molecule is in an amount effective to raise antibodies that specifically recognize adherent erythrocytes in a subject. Such compositions also are useful in the therapeutic methods of this invention. Such nucleic acids can also be used to raise antibodies in an animal for passive immunization of a subject.

The compositions of this invention can be prepared in unit dosage forms for administration to a subject. The amount and timing of administration are at the discretion of the treating physician to achieve the desired purposes.

The compositions of the present invention can be administered to reduce the number of adherent erythrocytes is a patient and, in malaria, to destroy infected erythrocytes, thereby reducing the proliferation of the parasite. In these applications, compositions are administered to a patient suffering from a disease which results in antibodies that specifically recognize adherent erythrocytes, in an amount sufficient to bind to and cause lysis of the adherent erythrocytes. Amounts effective for this use will depend upon the severity of the disease and the general state of the patient's health. An effective amount of the compound is that which provides either subjective relief of a symptom(s) or an objectively identifiable improvement as noted by the clinician or other qualified observer.

Single or multiple administrations of the compositions are administered depending on the dosage and frequency as required and tolerated by the patient. In any event, the composition should provide a sufficient quantity of the proteins of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

Controlled release parenteral formulations of the immunoconjugate compositions of the present invention can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., COLLOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp. 315-339, (1992) both of which are incorporated herein by reference.

Polymers can be used for ion-controlled release of immunoconjugate compositions of the present invention. Various degradable and nondegradable polymeric matrices for use in controlled drug delivery are known in the art (Langer, R., Accounts Chem. Res. 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston, et al., Pharm. Res. 9:425-434 (1992); and Pec, et al., J. Parent. Sci. Tech. 44(2):58-65 (1990)). Alternatively, hydroxyapatite has been used as a microcarrier for controlled release of proteins (Ijntema, et al., Int. J. Pharm. 112:215-224 (1994)). In yet another aspect, liposomes are used for controlled release as well as drug targeting of the lipid-capsulated drug (Betageri, et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

Either peptides of the invention or nucleic acids encoding them, or both, may also be administered via liposomes. Liposomes are useful in increasing the half-life of the peptides and in protecting the nucleic acids from extracellular nucleases. Liposomes include emulsions, foams, micelles, insoluble monolayers, liquid crystals, phospholipid dispersions, lamellar layers and the like. In liposome preparations, the peptide to be delivered may be incorporated as part of a liposome, alone or in conjunction with a molecule that binds to, e.g., a receptor prevalent among lymphoid cells, such as monoclonal antibodies that bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions.

In another embodiment, intact antibodies to peptides of the invention are administered to induce complement-dependent cytotoxicity or antibody-dependent, cell-mediated cytotoxicity of cells with conformational changes in AE1 exposing normally cryptic sequences of peptides of the invention. Conveniently, antibodies may be provided in lyophilized form and rehydrated with sterile water before administration, although they can also be provided in sterile solutions of known concentration. The antibody solution is then added to an infusion bag containing 0.9% Sodium Chloride, USP, and typically administered at a dosage of from 0.5 to 15 mg/kg of body weight. Considerable experience is available in the art in the administration of antibody drugs, which have been marketed in the U.S. at least since the approval of Rituxan® in 1997. Antibody drugs are desirably administered by slow infusion, rather than in an IV push or bolus. Typically, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Administration of nucleic acids, such as DNA and mRNA, to raise antibodies to an immunogen by introducing sequences that encode the immunogen, are known in the art. See, e.g., U.S. Pat. Nos. 5,703,055, 6,586,409, 5,703,055, 5,693,622, and 5,589,466. Devices to introduce such nucleic acids into the skin, muscle, or other tissue for expression are also known. See, e.g., U.S. Pat. No. 5,865,796.

### EXAMPLES

### Example 1

### Effect on DBR in Inhibiting Cytoadhesion and Recognition of DBR by Sera from Infected Patients

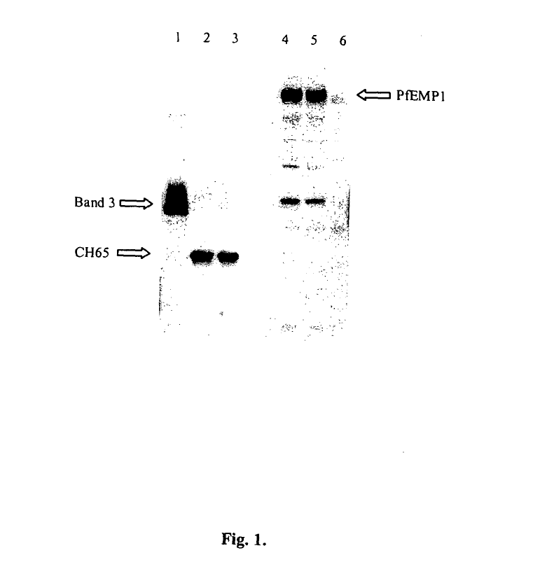

To determine the precise contribution of erythrocyte membrane proteins to the adherence of malaria infected erythrocytes, AE1 was specifically modified using several chemical reagents. As previously reported, AE1 can be efficiently cleaved by chymotrypsin treatment of intact human erythrocytes (FIG. 1). Malaria parasites invaded and developed normally into these chymotrypsinized erythrocytes as judged by Giemsa stained thin smears. To test whether residual chymotrypsin could be cleaving other surface exposed proteins such as the parasite encoded Plasmodium falciparum erythrocyte membrane protein 1 (PfEMP1), Western blot analysis was carried out. As shown in FIG. 1 (lanes 4-6) there are equivalent amounts of PfEMP1 in both chymotrypsin treated and untreated erythrocytes. In addition, there is no degradation of PfMP1 as the parasite develops from the ring to the mature trophozoite stage.

The binding capacity of chymotrypsin treated IEs to immobilized CD36 and thrombospondin (TSP) was tested. As shown in Table 1, there is a significant reduction in the degree of binding of IEs for CD36 but infected red cell binding to TSP was largely unaffected.

To further confirm the central role of AE1 on adherence, a highly specific modifying reagent was investigated. DIDS (4,4′-diisothiocyanatostilbene-2,2′-disulfonic acid) is a bifunctional inhibitor of anion exchange in erythrocytes and reacts covalently with Lys-539 in AE1. DIDS modified erythrocytes can also support the normal growth of the malaria parasite in vitro (not shown). Similar to chymotrypsin treatment, DIDS treated IEs show lower binding to CD36 but not to TSP (Table 1). Fluorescein maleimide (FMA) another potent anion exchange inhibitor of AE1 that reacts with Lys 430 did not reduce the binding of IEs to either CD36 or TSP (Table 1).

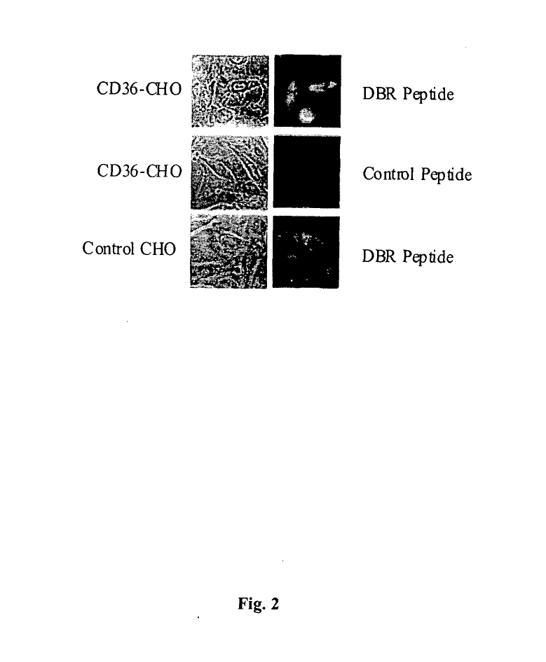

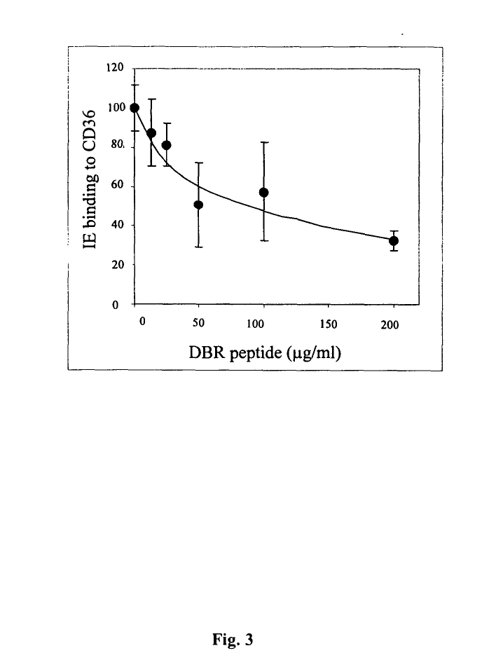

To confirm that the DIDS binding region (DBR) of AE1 is involved in IE adhesion, two different kinds of in vitro assays were carried out. Peptides derived from the DBR of human AE1 interacted with high affinity and specificity with CD36 transfected CHO cells (FIG. 2); and in a competitive binding assay DBR peptides inhibited the binding of IEs to immobilized CD36 (FIG. 3).

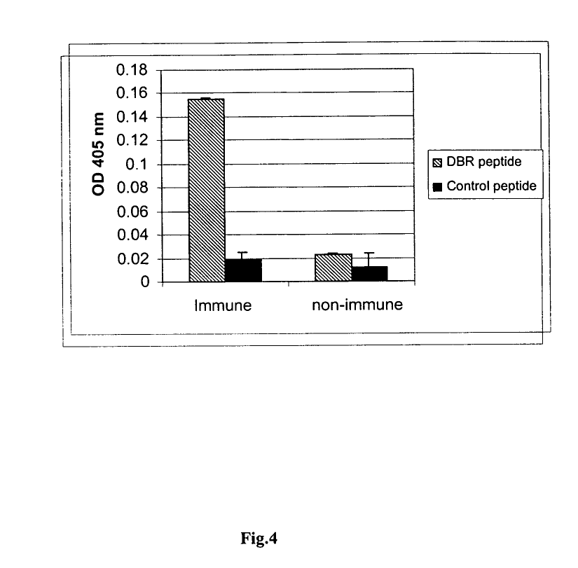

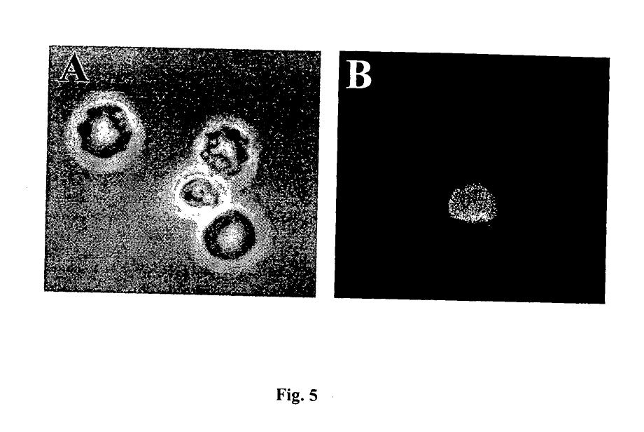

To explore whether DBR is exposed as a consequence of parasite development, sera from individuals living in a region where malaria is endemic or from a control group was incubated with intact live IEs and the surface bound immunoglobulins eluted by a low pH treatment. Using an ELISA, it was determined that individuals living in an endemic area (and without clinical signs of disease) presented a strong humoral immune response against the DBR (FIG. 4). In addition, polyclonal antibodies developed against synthetic peptides corresponding to the DBR recognized live IEs as shown by an indirect immunofluorescence assay (FIG. 5).

### Example 2

### Discussion of Results of Example 1

The role of the erythrocyte anion exchanger AE1 in the adhesion of P. falciparum infected erythrocytes (“IEs”) to CD36 was studied. A substantial reduction in the adhesive properties of IEs was observed when infected red cells were treated with specific proteases. The CD36 interaction with IE was exquisitely sensitive to chymotrypsin treatment, indicating that host cell membrane components play a substantial role in adhesion. In addition, chymotrypsin treated IEs could still bind to TSP. Our laboratory has previously demonstrated that the TSP interaction with IEs is mediated both by PS exposure and through a region of AE1 which is predicted to be unaffected by chymotrypsin digestion.

The relative contribution of PfEMP1 to IE adhesion is difficult to ascertain, but it appears that the role of this parasite protein in IE binding to CD36 may be less than previously claimed. Nevertheless, it is possible that large membrane structural changes caused by extensive protease treatment, could affect ligand-receptor interactions in an indirect manner. For example, by improper targeting/trafficking of PfEMP1 to the membrane or alternatively by an altered disposition/distribution of the parasite protein on the surface of the modified IE membrane.

Despite this, chemical treatment with DIDS, a reagent known to be highly selective for AE1 of human erythrocytes, provides persuasive evidence that a limited modification of AE1 does have a significant effect on IE adherence.

Finally, since both immunoglobulins from individuals living in malaria endemic regions and polyclonal antibodies developed against peptides corresponding to the DBR region were found to bind specifically to the surface of live infected erythrocytes, it is clear that intracellular development of the parasite results in exposure of cryptic regions of AE1.

The evidence presented in this study indicates that cytoadherence and neoantigen exposure in falciparum malaria result from parasite-induced modifications of the host red blood cell membrane protein, AE1.

TABLE 1
Effect of AE1 modification on the binding of infected erythrocytes to
CD36 or TSP.

| | CD36 | TSP |
|---|---|---|
| Chymotrypsin | 10.2 + 17.5 | 81.0 + 17.7 |
| DIDS | 33.2 + 4.1 | 93.5 + 35.9 |
| FMA | 135.1 + 12.9 | 180.9 + 26.0 |

Uninfected erythrocytes were treated with either 0.25 mg/ml fluorescein maleimide (FMA) (1 hr at 25° C.), 100 μM DIDS (1 hr at 37° C.) or with 0.5 mg/ml chymotrypsin (0.5 hr at 37° C.). Modified erythrocytes were infected and after 40 hr, mature stages were harvested and a standard cytoadherence assay using CD36 or TSP immobilized on slides was carried out. The numbers are the percentage of bound IEs/mm² as compared to untreated cells. Each value represents the mean ±SD of triplicate determinations.

### Example 3

### Use of DBR as an Immunogen Results in Antibodies that Destroy IE, but not Uninfected RBC

New Zealand White rabbits were immunized with 200 μg of DBR peptide conjugated to KLH in complete Freund's adjuvant, followed by 5 booster injections of 100 μg conjugated peptide in incomplete Freund's adjuvant. Pre-immune (pre) or immune (immune) sera was diluted in PBS and incubated for 1 hr at 37° C. with either uninfected erythrocytes (nrbc) or human erythrocytes infected with P. falciparum. Cells were centrifuged and the hemoglobin released into the supernatant was quantified by measuring the optical density at 405 nm.

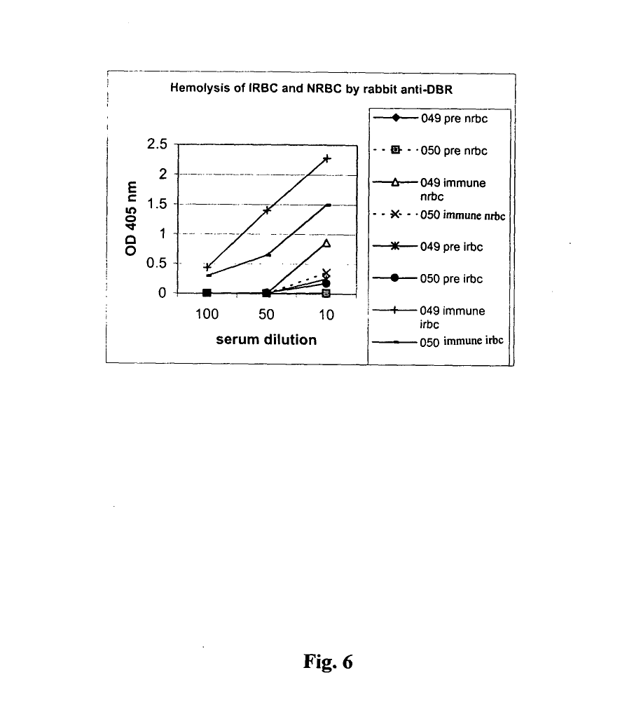

The results are shown in FIG. 6. The rabbits are designated as “049” and “050.” Serum from rabbits prior to immunization with DBR peptide is designated as “pre,” while serum from rabbits after immunization with the peptide is described as “immune.” Uninfected erythrocytes are designated as “nrbc.” At dilutions of 1:50 and 1:100, the immune sera recognized and destroyed infected erythrocytes, but not uninfected erythrocytes.

### Example 4

### Cytoadherence Assays used in the Studies Herein

Partially purified CD36 (CD36 fraction) was obtained from human platelets essentially by the method of Tandon et al., J. Biol Chem, 264:7570 (1989). However, the last purification step, using Ultrogel AcA-44, was omitted. TSP was purified as described in Layler et al., J Biol Chem, 253:8609 (1978). To remove excess amount of Triton X-100 in CD36 solution, 5 ml of a CD36 solution containing 0.1% Triton X-100 (initial concentration 268 μg protein/ml) was incubated with 0.5 g of SM-2 beads (Bio-Rad, Hercules, Calif.) at room temperature for 2 h and dialyzed against 500 ml of distilled water. Five microliters of the dialyzed solution was mounted onto multi-well glass slides, and incubated at 4° C. overnight. To immobilize TSP, 5 μl of TSP solution (50 μg/ml in 50 mM Bis-Tris, 100 mM NaCl, 25 mM calcium lactate, pH 7.4 (BTC)) was spotted on plastic slides (Permanox® plastic, Nunc, Neperville, Ill.) and incubated at 4° C. overnight. After removal of the solution by gentle aspiration, CD36- and TSP-immobilized spots were blocked with 1% BSA in phosphate buffered saline (PBS) at room temperature for 1 h.

Cytoadherence assays were performed as described in Crandall et al., Exp Parasitol, 73:362 (1991) (“Crandall 1991”). In the case of inhibition assay using peptides, the spots of immobilized protein were pretreated with 10 μl of peptide solution (200 μg/ml) in Bis-Tris buffer (50 mM Bis-Tris, 100 mM NaCl, 2 mM CaCl₂, pH 6.6 for CD36; 50 mM Bis-Tris, 100 mM NaCl, 2 mM CaCl₂, pH 7.4 for 677 cells; BTC for TSP) at 37° C. for 1 h. After removal of the peptide solution, the slides were placed in individual compartments of a partitioned plastic box containing 7 ml Bis-Tris buffer. Fifty microliters of packed red cells (5-10% parasitemia) was added to each compartment of the plastic box. After incubation at 37° C. for 90 min with gentle rocking, the red cell suspension was carefully aspirated. The slides were rinsed by adding 7 ml of the buffer and 5 min of rocking after which time the buffer was aspirated. The slides were washed three times, fixed, and stained as described in Crandall 1991. The number of bound infected red cells/mm² area of immobilized protein was counted by light microscopy.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1: A use of an isolated peptide of 40 or fewer amino acids, comprising a sequence with at least 90% identity to a sequence TFSX₁LIX₂IFQ (SEQ ID NO:4), where X₁ and X₂ are independently selected from amino acids with a charge under physiological conditions, and wherein said peptide, when presented as an antigen, raises antibodies which bind to and cause destruction of pathologically adherent erythrocytes, for the manufacture of a medicament to cause destruction of erythrocytes that adhere to vascular endothelial cells due to a pathological condition.

2: A use of claim 1, wherein X₁ and X₂ are both negatively charged.

3: A use of claim 1, wherein X₁ and X₂ are both positively charged.

4: A use of claim 1, wherein X₁ and X₂ are both lysine.

5: A use of claim 1, wherein one or more of said amino acids is a D-amino acid.

6: A use of claim 1, wherein said peptide has the sequence TFSKLIKIFQ (SEQ ID NO:3).

7: A use of claim 1, wherein said pathological condition is selected from the group consisting of diabetes, thalassemia, sickle cell anemia, and malaria.

8: A use of claim 1, wherein said medicament comprises antibodies.

9: A use of claim 8, wherein said antibodies are polyclonal.

10: A use of claim 8, wherein said antibodies are monoclonal.

11: A use of claim 10, wherein said monoclonal antibodies are humanized.

12: A use of a nucleic acid encoding an isolated peptide of 40 or fewer amino acids, comprising a sequence at least 90% identical to a sequence TFSX₁LIX₂IFQ (SEQ ID NO:4), where X₁ and X₂ are independently selected from amino acids with a charge under physiological conditions, and wherein antibodies raised by said peptide bind to and cause destruction of pathologically adherent erythrocytes, for the manufacture of a medicament to cause destruction of erythrocytes that adhere to vascular endothelial cells due to a pathological condition.

13: A use of claim 12, wherein X₁ and X₂ are both negatively charged.

14: A use of claim 12, wherein X₁ and X₂ are both positively charged.

15: A use of claim 12, wherein X₁ and X₂ are both lysine.

16: A use of claim 12, wherein said pathological condition is selected from the group consisting of diabetes, thalassemia, sickle cell anemia, and malaria.

17: A method for lysing erythrocytes adherent due to a pathological condition, said method comprising administering to a patient with said adherent erythrocytes antibodies that specifically bind to a protein having an amino acid sequence YETFSKLIKIFQDH (SEQ ID NO:5) on said erythrocytes, wherein binding of said antibodies to said amino acid sequence results in destruction of said adherent erythrocytes.

18: A method of claim 17, wherein said pathological condition is selected from the group consisting of diabetes, thalassemia, sickle cell anemia, and malaria.

19: A method for lysing erythrocytes adherent due to a pathological condition, said method comprising administering to a patient with said pathologically adherent erythrocytes an isolated peptide with at least 80% sequence identity to a sequence YX₁TFSX₂LIX₃IFQX₄X₅ (SEQ ID NO:6), or a fragment thereof, which peptide or fragment thereof, when presented as an antigen, raises antibodies which specifically bind to and cause destruction of said pathologically adherent erythrocytes, wherein X₁, X₂, X₃, X₄ and X₅ are independently selected from amino acids that bear a charge at physiological pH.

20: A method of claim 19, wherein X₁ and X₄ bear the same charge and X₂ and X₃ bear the same charge, but the charge borne by X₁ and X₄ is not the same as the charge borne by X₂ and X₃.

21: A method of claim 20, wherein the charge borne by X₂ and X₃ is positive.

22: A method of claim 19, wherein X₂ and X₃ are lysine residues.

23: A method of claim 19, wherein said peptide has 100% sequence identity to SEQ ID NO:6 and further wherein X₂ and X₃ are lysine residues, X₁ is a glutamic acid, X₄ is an aspartic acid and X₅ is a histidine (SEQ ID NO:5).

24: A method of claim 19, wherein one or more of said amino acids is a D-amino acid.

25: A method for lysing erythrocytes adherent due to a pathological condition, said method comprising administering to a patient with said pathologically adherent erythrocytes a nucleic acid encoding a peptide with at least 80% sequence identity to the sequence YX₁TFSX₂LIX₃IFQX₄X₅ (SEQ ID NO:6), or fragment thereof which raises antibodies which specifically recognize said peptide, wherein X₁, X₂, X₃, X₄, and X₅ are independently selected from amino acids that bear a charge at physiological pH, wherein expression of said peptide raises antibodies which specifically bind to and cause destruction of said pathologically adherent erythrocytes.

26: A method of claim 25, wherein X₁ and X₄ bear the same charge and X₂ and X₃ bear the same charge, but the charge borne by X₁ and X₄ is not the same as the charge borne by X₂ and X₃.

27: A method of claim 25, wherein the charge borne by X₂ and X₃ is positive.

28: A method of claim 25, wherein X₂ and X₃ are lysine residues.

29: A method of claim 25, wherein said peptide has 100% sequence identity to SEQ ID NO:6 and further wherein X₂ and X₃ are lysine residues, X₁ is a glutamic acid, X₄ is an aspartic acid and X₅ is a histidine (SEQ ID NO:5).

30: A composition of an isolated peptide of the formula with at least 80% sequence identity to a sequence YX₁TFSX₂LIX₃IFQX₄X₅ (SEQ ID NO:6), wherein X₁, X₂, X₃, X₄ and X₅ are independently selected from amino acids that bear a charge at physiological pH, and wherein antibodies raised by said peptide bind to and cause destruction of pathologically adherent erythrocytes, and a pharmaceutically acceptable carrier.

31: A composition of claim 30, wherein X₁ and X₄ bear the same charge and X₂ and X₃ bear the same charge, but the charge borne by X₁ and X₄ is not the same as the charge borne by X₂ and X₃.

32: A composition of claim 30, wherein the charge borne by X₂ and X₃ is positive.

33: A composition of claim 30, wherein X₂ and X₃ are lysine residues.

34: A composition of claim 30, wherein said peptide has 100% sequence identity to SEQ ID NO:6 and further wherein X₂ and X₃ are lysine residues, X₁ is a glutamic acid, X₄ is an aspartic acid, and X₅ is a histidine (SEQ ID NO:5).

35: A composition of claim 30, wherein one or more of said amino acids is a D-amino acid.

36: An isolated peptide with at least 80% sequence identity to the sequence YX₁TFSX₂LIX₃IFQX₄X₅ (SEQ ID NO:6) or fragment thereof, which peptide or fragment, when presented as an antigen, raises antibodies that specifically bind to SEQ ID NO:5 and cause destruction of pathologically adherent erythrocytes and wherein X₁, X₂, X₃, X₄ and X₅ are independently selected from amino acids that bear a charge at physiological pH.

37: An isolated peptide of claim 36, wherein X₁ and X₄ bear the same charge and X₂ and X₃ bear the same charge, but the charge borne by X₁ and X₄ is not the same as the charge borne by X₂ and X₃.

38: An isolated peptide of claim 36, wherein the charge borne by X₂ and X₃ is positive.

39: An isolated peptide of claim 36, wherein X₂ and X₃ are lysine residues.

40: An isolated peptide of claim 36, which peptide has 100% sequence identity to SEQ ID NO:6 and further wherein X₂ and X₃ are lysine residues, X₁ is a glutamic acid, X₄ is an aspartic acid and X₅ is a histidine (SEQ ID NO:5).

41: An isolated peptide of claim 36, wherein one or more of said amino acids is a D-amino acid.

42: An isolated nucleic acid encoding a peptide with at least 80% sequence identity to YX₁TFSX₂LIX₃IFQX₄X₅ (SEQ ID NO:6) or a fragment thereof, which peptide or fragment, when presented as an antigen, raises antibodies that specifically bind to SEQ ID NO:5 and cause destruction of pathologically adherent erythrocytes and further wherein X₁, X₂, X₃, X₄, and X₅ are independently selected from amino acids that bear a charge at physiological pH.

43: An isolated nucleic acid of claim 42, wherein X₁ and X₄ bear the same charge and X₂ and X₃ bear the same charge, but the charge borne by X₁ and X₄ is not the same as the charge borne by X₂ and X₃.

44: An isolated nucleic acid of claim 42, wherein the charge borne by X₂ and X₃ is positive.

45: An isolated nucleic acid of claim 42, wherein X₂ and X₃ are lysine residues.

46: An isolated nucleic acid of claim 42, wherein said encoded peptide has 100% sequence identity to SEQ ID NO:6 and further wherein X₂ and X₃ are lysine residues, X₁ is a glutamic acid, X₄ is an aspartic acid, and X₅ is a histidine (SEQ ID NO:5).

47: An isolated nucleic acid of claim 42 operably linked to a promoter.

48: An isolated nucleic acid of claim 46 operably linked to a promoter.

49: A composition of an isolated nucleic acid encoding a peptide with at least 80% sequence identity to the sequence YX₁TFSX₂LIX₃IFQX₄X₅ (SEQ ID NO:6) or fragment thereof, which peptide or fragment, when presented as an antigen, raises antibodies that specifically bind to SEQ ID NO:5 and cause destruction of pathologically adherent erythrocytes, wherein X₁, X₂, X₃, X₄, and X₅ are independently selected from amino acids that bear a charge at physiological pH, and a pharmaceutically acceptable carrier.

50: A composition of claim 49, wherein X₂ and X₃ are lysine residues, X₁ is a glutamic acid, X₄ is an aspartic acid, and X₅ is a histidine (SEQ ID NO:5).
